(19) United States
(12) Patent Application Publication
(10) Pub. No.: US 2021/0361175 A1
(43) Pub. Date: Nov. 25, 2021

(54) SLEEP-BASED BIOMETRIC TO PREDICT AND TRACK VIRAL INFECTION PHASES

(71) Applicant: KONINKLIJKE PHILIPS N.V.

(72) Inventors: Gary Nelson GARCIA MOLINA, Madison, WI (US); Xia CHEN, Lorton, VA (US); Yash Parag MOKASHI, Pittsburgh, PA (US)

(21) Appl. No.: 17/218,234

(22) Filed: Mar. 31, 2021

Related U.S. Application Data
(60) Provisional application No. 63/027,406, filed on May 20, 2020.

(51) Int. Cl. A61B 5/0205; A61B 5/00; A61B 5/024; A61B 5/369; A61B 5/11; A61B 5/05; A61B 5/08

(52) U.S. Cl. CPC A61B 5/02055; A61B 5/7275; A61B 5/4809; A61B 5/02405; A61B 5/369; A61B 5/1102; A61B 5/7278; A61B 5/02416; A61B 5/08; A61B 5/1118; A61B 5/7264; A61B 5/4812; A61B 5/05

(57) ABSTRACT

An apparatus and method involve leveraging unobtrusive sleep monitoring technologies, even consumer ones, to predict the phase of a viral infection and is usable for guiding patient treatment and tracking treatment effectiveness. An apparatus and method determine a phase of a viral infection based at least in part upon a data set that is input to an algorithm.

### CROSS-REFERENCE TO PRIOR APPLICATIONS

This application claims the benefit of U.S. Provisional Application No. 63/027,406, filed on 20 May 2020. This application is hereby incorporated by reference herein.

### BACKGROUND OF THE INVENTION

### 1. Field of the Invention

The present invention pertains to enabling the guiding of patient treatment and the tracking of treatment effectiveness during a pandemic and otherwise and, in particular, to an apparatus and method for leveraging unobtrusive sleep monitoring technologies, even consumer ones, to predict the phase of the viral infection.

### 2. Description of the Related Art

Sleep and the circadian system exert a strong regulatory influence on immune functions. For instance the production of pro-inflammatory cytokines peaks during early nocturnal Non-Rapid Eye Movement (NREM) sleep, whereas the quantity of natural killer cells and the anti-inflammatory cytokine activity peak during daytime wakefulness. L. Besedovsky, T. Lange, and J. Born, “Sleep and immune function,” Pflugers Arch. Eur. J. Physiol., vol. 463, no. 1, pp. 121-137, 2012.

A conceptual model of the effect of immunity-function activation on sleep is presented in FIG. 1, which depicts a conceptual model of sleep changes in response to immune activation and underlying mechanisms. FIG. 1 is inspired by L. Besedovsky, T. Lange, and M. Haack, “The sleep-immune crosstalk in health and disease,” Physiol. Rev., vol. 99, no. 3, pp. 1325-1380, July 2019.

Environmental stimuli including certain edibles, stress, bacteria, and/or viruses activate the immune system by exhibiting molecular patterns referred to as Damage Associated Molecular Patterns (DAMPs) and/or Pathogen Associated Molecular Patterns (PAMPs). DAMPS and PAMPs tend to activate Pattern Recognition Receptors (PRRs) which induce inflammatory responses including interleukin (IL)-1 and Tumor Necrosis Factor (TNF). The latter are NREM sleep promoting substances. According to the model proposed by Besedovksky et al., supra, subtle immune activation may be involved in homeostatic NREM sleep regulation that in turn could serve to restore immune homeostasis. More pronounced immune activation during an infection can induce a sleep response that, in turn, may support host defense and immunological memory formation. However, an extreme immune activation (e.g., during severe infection) seems to disrupt both NREM and Rapid Eye Movement (REM) sleep, often accompanied by sleep fragmentation, feelings of nonrestorative sleep, and daytime fatigue.

The outcome of the virus-host encounter often depends on the virulence of the infecting virus and the susceptibility of the host. J. MacLachlan and E. Dubovi, “Pathogenesis of Viral Infections and Diseases,” in Fenner's Veterinary Virology, Elsevier, 2017, pp. 47-78. Virulence refers to a quantitative or relative measure of the pathogenicity of the infecting virus. That is, a virus is said to be either pathogenic or nonpathogenic, but its virulence is stated in relative terms (e.g. “virus A is more virulent than virus B”). The virulence of a particular virus, administered in a particular dose, by a particular route, to a particular age and strain, may be assessed by determining its ability to cause disease, death, specific clinical signs, or lesions. The dose of the virus required to cause death in 50% of animals (Lethal Dose 50, LD50) has been a commonly used measure of virulence. Host susceptibility to viral diseases is affected by innate and adaptive immune responses as well as age, nutritional status, and cell differentiation.

The particular type of viruses (e.g. SARS-CoV-1 and -2) that enter the host mostly via the respiratory tract can cause an early inflammatory response because the respiratory system is protected by innate and adaptive immune mechanisms which operate at all mucosal surfaces. As mentioned elsewhere herein, inflammatory responses that are mediated by cytokines tend to modify sleep.

The severe acute respiratory syndrome coronavirus 2 (SARS-CoV-2) associated coronavirus disease 2019 (COVID-19) gripped the world in an unprecedented pandemic. Pharmacotherapy targeted against the virus appeared to be the most promising when applied early in the course of the illness, but its usefulness in advanced phases is more uncertain. M. Wang et al., “Remdesivir and chloroquine effectively inhibit the recently emerged novel coronavirus (2019-nCoV) in vitro,” Cell Res., vol. 30, no. 3, pp. 269-271, March 2020. Similarly, use of anti-inflammatory therapy applied too early may not be necessary and could even provoke viral replication, such as in the case of corticosteroids. C. D. Russell, J. E. Millar, and J. K. Baillie, “Clinical evidence does not support corticosteroid treatment for 2019-nCoV lung injury,” Lancet, vol. 395, no. 10223, pp. 473-475, February 2020. Two distinct but overlapping pathological states can be identified. The first is triggered by the virus itself, and the second is triggered by the host response. Siddiqi et al. 2020 propose a three-phase model that is illustrated in Error! Reference source not found., which depicts classification of disease states and clinical signs associated with COVID-19 pathology. FIG. 2 thus is adapted from H. K. Siddiqi and M. R. Mehra, “COVID-19 Illness in Native and Immunosuppressed States: A Clinical-Therapeutic Staging Proposal,” J. Hear. LUNG Transplant., vol. Preprint, 2020.

### Phase 1—Early Infection

The initial phase occurs at the time of inoculation and early establishment of disease. For most people, this involves an incubation period associated with mild and often non-specific symptoms such as malaise, fever, and a dry cough. During this period, SARS-CoV-2 multiplies and establishes residence in the host, primarily focusing on the respiratory system.

SARS-CoV-2 binds to its target using the Angiotensin-Converting Enzyme 2 (ACE2) receptor on human cells. These receptors are abundantly present on human lung and small intestine epithelium, as well as the vascular endothelium. Treatment at this phase is primarily targeted towards symptomatic relief. Should a viable anti-viral therapy (such as Remdesivir) be proven beneficial, the targeting of selected patients during this phase may reduce the duration of symptoms, minimize contagiousness, and prevent progression of severity. H. K. Siddiqui et al., supra.

Phase 2—Pulmonary Involvement with/without Hypoxia

In the second phase of established pulmonary disease, viral multiplication and localized inflammation in the lung is the norm. During this phase, patients develop a viral pneumonia, with cough, fever, and possibly hypoxia (defined as a PaO₂/FiO₂ of <300 mmHg). Markers of systemic inflammation may be elevated, but not remarkably so. It is at this phase that most patients with COVID-19 would need to be hospitalized for close observation and management. Treatment would primarily consist of supportive measures and available anti-viral therapies such as Remdesivir.

### Phase 3—Systemic Hyperinflammation

A minority of COVID-19 patients will transition into the third and most severe phase of illness, which manifests as an extra-pulmonary systemic hyperinflammation syndrome. In this phase, markers of systemic inflammation appear to be elevated. Tailored therapy in phase 3 hinges on the use of immunomodulatory agents to reduce systemic inflammation before it overwhelmingly results in multi-organ dysfunction. In this phase, use of corticosteroids may be justified in concert with the use of cytokine inhibitors.

As such, improvements in treatment of a viral infection, such as in the event of a pandemic and otherwise, would be desirable.

### SUMMARY OF THE INVENTION

Accordingly, it is an object of the present invention to provide an improved apparatus and method for leveraging sleep monitoring technologies, potentially including such technologies that are unobtrusive, even consumer ones, to predict the phase of a viral infection that overcomes the shortcomings of conventional systems and methods for guiding patient treatment and tracking treatment effectiveness. This object is achieved according to one embodiment of the present invention by providing an apparatus and method that determine a phase of a viral infection based at least in part upon a data set that is input to an algorithm.

Major recent disease outbreaks have been caused by viral infections, such as Severe Acute Respiratory Syndrome (SARS), Middle East Respiratory Syndrome (MERS), Ebola, and Covid-19. Treating viral conditions is difficult due to fast mutations and the fact that the optimal medication depends on the phase of the viral infection, i.e., attachment, incubation, symptomatic, inflammation.

Immunological activation caused by viral infections affects several aspects of sleep architecture and micro-events thereof. This invention leverages sleep monitoring technologies, including unobtrusive sleep monitoring technologies, even consumer ones, to derive a score that predicts the phase of the viral infection. This prediction guides treatment, and tracks treatment effectiveness.

Treating viral infections with the appropriate pharmacological agent thus depends upon whether the phase of the illness can be accurately detected. It is particularly useful to distinguish the phase where the viral pathogenicity is dominant versus when the host inflammatory response overtakes the pathology. It is also evident that early detection of the condition favors positive prognosis inasmuch as the condition can be controlled at an early phase, thus resisting a transition of the condition into the inflammatory response phase.

The need for administering pharmacological therapy at the appropriate phase has been recently evidenced for the particular case of COVID-19 where a trial involving a promising immunomodulatory drug hydroxychloroquine (J. Magagnoli et al., “Outcomes of hydroxychloroquine usage in United States veterans hospitalized with Covid-19,” medRxiv, p. 2020.04.16.20065920, April 2020) revealed that this drug may not be effective if administered in late phases of the illness. The importance of administering a pharmacological agent in a timely and early fashion in viral infections has also been emphasized in the case of SARS and MERS. A. Zumla, J. F. W. Chan, E. I. Azhar, D. S. C. Hui, and K. Y. Yuen, “Coronaviruses-drug discovery and therapeutic options,” Nat. Rev. Drug Discov., vol. 15, no. 5, pp. 327-347, May 2016.

Continuous tracking of the phases of the viral illness to guide treatment and to permit the tracking of the effectiveness of the treatment is desirable and can more effectively be accomplished using signals that are unobtrusively and continuously acquired. This invention proposes to use sleep monitoring to build a score that reflects the phase of the viral illness. For instance, the phase of the viral illness is a categorical value: 1, 2, or 3 whereas the score S is a numerical value that will provide a probability associated with the phase of the viral illness and be compared against thresholds to determine the phase of the illness.

The basic concept of this invention consists in utilizing the sleep architecture of a patient to build an index that estimates the phase of the pathology, i.e., the phase of the viral infection, in the patient. Knowing this information can advantageously inform a medical team about the phase of the patient, enable early detection of the conditions, and guide the pharmacological treatment.

In the disclosed and claimed concept, the sleep of the patient is monitored using consumer type of sensors including a ballistocardiography device (R. Yi, M. Enayati, J. M. Keller, M. Popescu, and M. Skubic, “Non-Invasive In-Home Sleep Stage Classification Using a Ballistocardiography Bed Sensor,” in IEEE-EMS International Conference On Biomedical And Health Informatics, 2019, no. July, pp. 1-4.), a radar device (V. P. Tran, A. A. Al-Jumaily, and S. M. S. Islam, “Doppler Radar-Based Non-Contact Health Monitoring for Obstructive Sleep Apnea Diagnosis: A Comprehensive Review,” Big Data Cogn. Comput., vol. 3, no. 1, p. 3, January 2019.), a device that detects cardiac signals; e.g. using a photoplethysmography bracelet (P. Fonseca et al., “Validation of Photoplethysmography-Based Sleep Staging Compared With Polysomnography in Healthy Middle Aged Adults,” Sleep, vol. 3, 2017), or a device that detects an electroencephalogram (G. Garcia-Molina et al., “Closed-loop system to enhance slow-wave activity,” J. Neural Eng., vol. 15, no. 6, pp. 1-11, 2018). It is also possible to combine the individual input from one or more of these devices to increase the overall accuracy.

In the disclosed and claimed concept, a patient's sleep architecture, i.e. percent of sleep states, duration of sleep states, etc., are aggregated into a score that can be used to characterize the phase of the viral condition in the patient. In phase 1, the patient's current duration of NREM sleep increases compared with the a baseline duration of NREM sleep because of the sleep-promoting cytokines, however in phase 1 the REM sleep duration remains comparable to that of the baseline. In this regard, it is noted that baseline may refer to the patient's own baseline, i.e., based upon prior data from the patient, or it may be based upon a population to which the patient belongs, or both.

In phase 2, NREM sleep duration increases further and REM sleep duration decreases. In phase 3, resolution of the illness will translate into normalization of the NREM to REM sleep proportions whereas exacerbation of the illness will result in sleep disruption of both NREM and REM sleep.

Accordingly, aspects of the disclosed and claimed concept are provided by an improved method of determining a phase from among a plurality of phases of a viral infection in a patient, the general nature of which can be stated as including inputting to an algorithm a data set that comprises a set of parameters that are representative of the patient's current sleep architecture and another set of parameters that are representative of a baseline sleep architecture, and determining the phase of the viral infection based at least in part upon the data set.

Other aspects of the disclosed and claimed concept are provided by an improved apparatus structured to determine a phase from among a plurality of phases of a viral infection in a patient, the general nature of which can be stated as including a processor apparatus comprising a processor and a storage, an input apparatus structured to provide input signals to the processor apparatus, an output apparatus structured to receive output signals from the processor apparatus and the storage having stored therein a number of routines which, when executed on the processor, cause the apparatus to perform operations the general nature of which can be stated as including inputting to an algorithm a data set that comprises a set of parameters that are representative of the patient's current sleep architecture and another set of parameters that are representative of a baseline sleep architecture, and determining the phase of the viral infection based at least in part upon the data set. As employed herein, the expression “a number of” and variations thereof shall refer broadly to any non-zero quantity, including a quantity of one.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

FIG. 1 is a conceptual model of the effect of immunity-function activation on sleep;

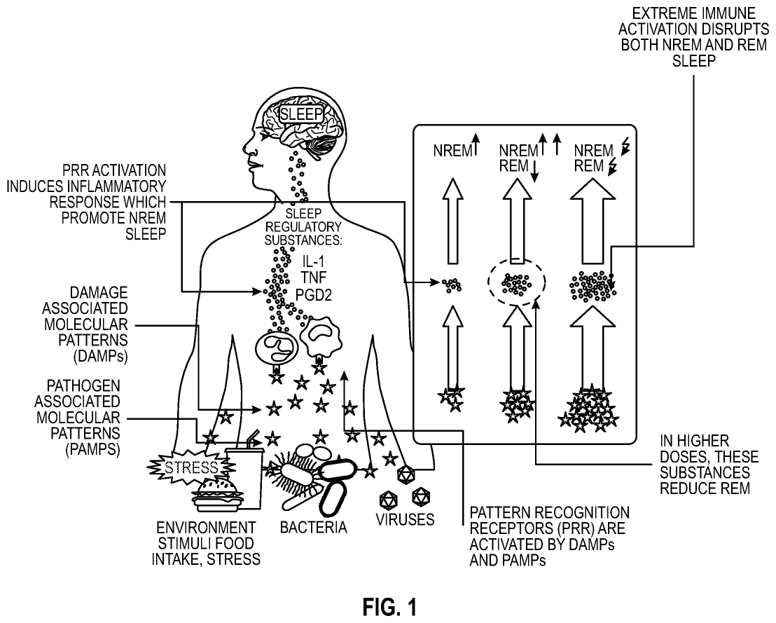

FIG. 2 is a three-phase model which depicts classification of disease states and clinical signs associated with at least one viral pathology;

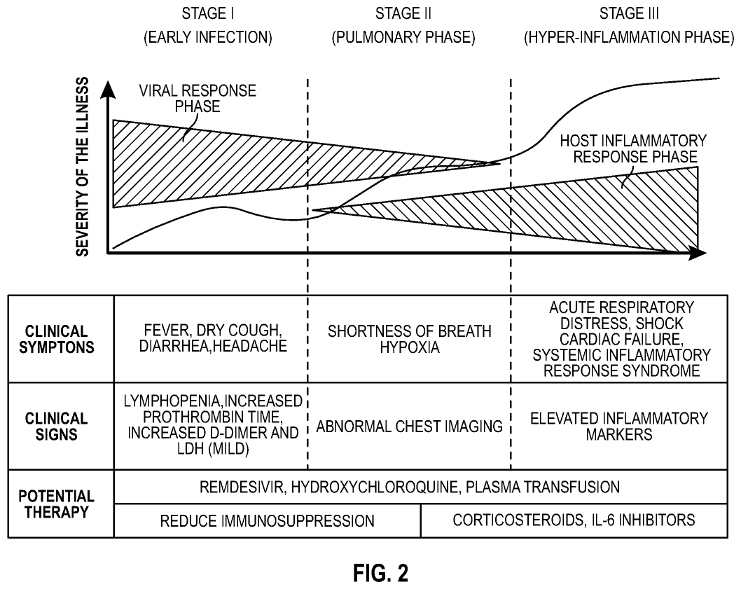

FIG. 3 is a depiction of a sleep-based biometric that are embodied in an improved method and apparatus in accordance with the disclosed and claimed concept to predict and track viral infection phases;

FIG. 4 is an exemplary depiction of at least a portion of a viral infection phase decision tree that can be implemented with a machine-learning system in accordance with the improved method and apparatus in accordance with the disclosed and claimed concept.

### DETAILED DESCRIPTION OF EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

The invention advantageously creates a model to evaluate the viral infection phase with an aggregated score through tracking and analyzing sleep metrics based on the following information. Denote the current duration of NREM sleep as D_{NREM}; denote the current duration of REM sleep as D_{REM}; denote the current duration of Wake After Sleep Onset (WASO) as D_{WASO}; and denote the current total sleep duration as D_{TST}. Likewise, denote the baseline (e.g. weekly average) of NREM sleep as D_{NREMB}; denote the baseline (e.g. weekly average) of REM sleep as D_{REMB}; denote the baseline (e.g. weekly average) of WASO as D_{WASOB}; and denote the baseline (e.g. weekly average) of total sleep duration as D_{TSTB}. Additionally, denote S as the aggregated score of a viral condition based on sleep biometrics. S will be in one of the following ranges:

TABLE 1
Aggregated Score Range and Viral Infection Phases

| Aggregated Score Range (S) | Viral Infection Phase |
|---|---|
| 0 | Baseline (no infection) |
| 1-100 | Phase 1 |
| 101-1000 | Phase 2 |
| 1001+ | Phase 3 |

The aggregated score S can be defined as a function of relevant sleep metrics that at least includes, but not limited to, NREM, REM, TST, and WASO. In the exemplary embodiment described herein, the aggregated score S can be represented as a function of percentage changes of these metrics,

S=W₁×F₁+W₂×F₂+W₃×F₃ where

F₁ is a function of % change of current NREM sleep as compared with baseline, e.g. F1=100×((D_{NREM}/D_{TST})−(D_{NREMB}/D_{TSTB}));

F₂ is a function of % change of current REM sleep as compared with baseline, e.g. F₂=100×((D_{REMB}/D_{TSTB})−(D_{REM}/D_{TST}));

F₃ is a function of % change of current WASO as compared with baseline, e.g. F₃=100×((D_{WASO}/D_{TST}) (D_{WASOB}/D_{TSTB})); and

where W₁, W₂, and W₃ are the corresponding weights that are applied to each function. A data set formed of the aforementioned parameters D_{NREM}; D_{REM}; D_{WASO}; D_{TST}; D_{NREMB}; D_{REMB}; D_{WASOB}; D_{TSTB} for a given patient could thus be used to determine a score for the patient which describes the phase of the viral illness in the patient. This can then be output to a medical team, or the output could be in the form of an instruction to provide a certain type of medical care, such an instruction to administer pharmacotherapy in the event that the score S indicates that the patient is early in the course of the illness, but or to provide anti-inflammatory therapy in the event that the score S indicates that the patient is later in the course of the illness.

In some embodiments, the weights can be pre-determined to distinguish the impact of each corresponding function. For instance, is W₁=100; W₂=1000; W₃=10000, this will result in the estimated score ranges being as defined in Table 1. To improve the accuracy of the estimation, a moving average of % changes (e.g. past 3 nights) instead of % change on a single night could be used for evaluation.

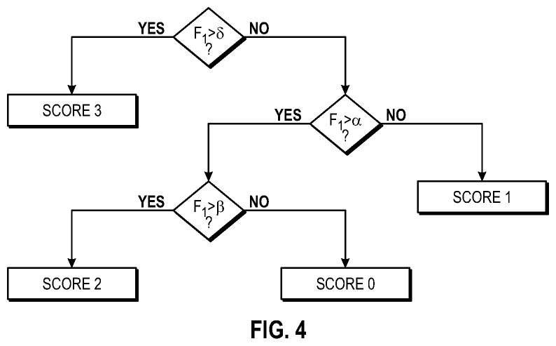

In one implementation, the disclosed and claimed concept is embodied in a decision tree based upon a machine learning algorithm to determine the viral infection phase through tracking and analyzing sleep metrics. An example of such a decision tree is depicted generally in FIG. 4 with the notations set forth elsewhere herein. With the decision tree, the estimated score is a discrete value that maps to a specific viral infection phase as in Table 2:

TABLE 2
Estimated Score and Viral infection Phase

| Estimated Score (S) | Viral Infection Phase |
|---|---|
| 0 | Baseline (no infection) |
| 1 | Phase 1 |
| 2 | Phase 2 |
| 3 | Phase 3 |

The initial percentage change thresholds α, β, and δ for the corresponding sleep metrics are selected heuristically. They can be improved through continuous learning approach (e.g. minimize the aggregated cost function) as the model is being used continuously in the large population.

By way of example, each patient could be characterized as a data set in the exemplary form of a vector, i.e., an array of values, such as the nine parameters D_{NREM}; D_{REM}; D_{WASO}; D_{TST}; D_{NREMB}; D_{REMB}; D_{WASOB}; D_{TSTB}; and S. The decision tree might ask a series of yes/no questions of the vector, and each answer would lead either to another question or to a decision. For instance, the questions might begin with something like “is D_{NREM} greater than 20?” If the answer to that question is “yes”, the next question in the decision tree might be something like “is D_{REM} less than 30?” The answer to this question would then lead to another question, and so forth, until a determination is made by the decision tree about what is the phase of the patient's viral illness. Responsive to this determination, the output can be in the form of the patient's score, the phase of the patient's viral illness, and/or an instruction to provide a certain therapy to the patient.

In an alternative embodiment, the presence of additional biometric data points, like respiratory rate, heart rate, body temperature, SpO₂ etc. can be used by the invention to provide early detection of a viral infection. If, in additional to the change in sleep architecture described elsewhere herein, at least one additional biometric data point falls beyond a normal range for a predetermined duration, it can be inferred that the user is at a higher susceptibility for a viral infection. The user is then alerted to take further action of either contacting their healthcare provider or consulting for a check-up to confirm the presence an infection. The previously stated embodiments can be used to determine the phase of infection and drive the appropriate treatment decisions.

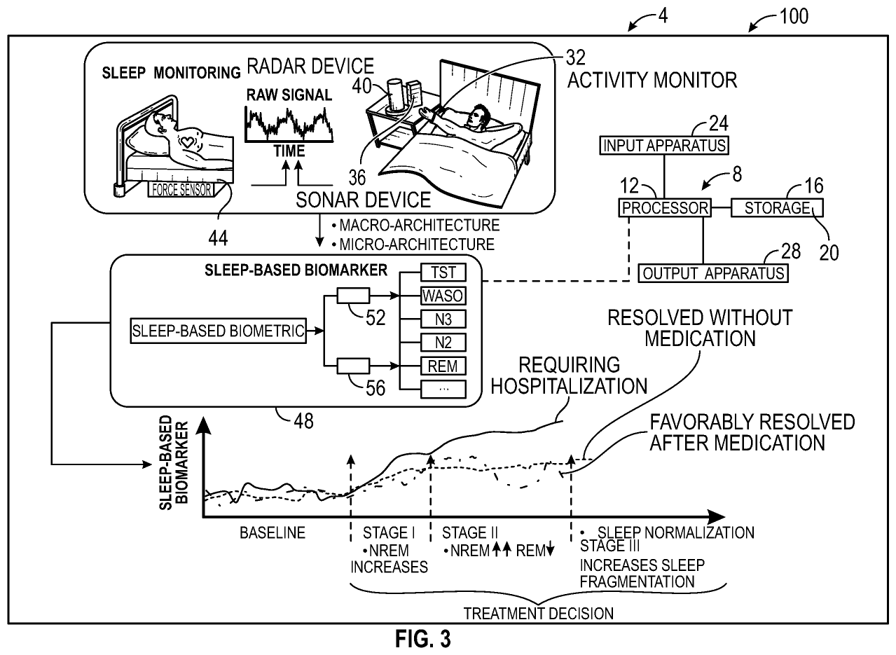

This invention is advantageously applicable to numerous known products. For instance Philips's Smart Sleep Deep Sleep Headband can provide numerous of the inputs described in FIG. 3. Existing Philips offerings track patients both in-hospital and at home. This solution can be utilized while monitoring patients during and after respiratory disease treatments, both in and out of hospital. This invention alone or in conjunction with patient monitoring can be utilized to advantageously provide important insights to customers (consumers, patients, and/or healthcare providers) in early detection and phase prediction of viral infections and in monitoring of recovery from viral infections. This invention is particularly useful for viral conditions that affect the respiratory system as these are characterized by inflammatory responses mediated by cytokines, the latter of which are well-known for regulating sleep.

An improved apparatus 4 in accordance with the disclosed and claimed concept is depicted in a schematic fashion in FIG. 3. Apparatus 4 can be employed in performing an improved method 100 that is likewise in accordance with the disclosed and claimed concept and at least a portion of which is depicted in a schematic fashion in FIG. 3. Apparatus 4 can be characterized as including a processor apparatus 8 that can be said to include a processor 12 and a storage 16 that are connected with one another. Storage 16 is in the form of a non-transitory storage medium that has stored therein a number of routines 20 that are likewise in the form of a non-transitory storage medium and that include instructions which, when executed on processor 12, cause apparatus 4 to perform certain operations such as are mentioned elsewhere herein.

Apparatus 4 further can be said to include an input apparatus 24 that provides input signals to processor 12 and to further include an output apparatus 28 that receives output signals from processor 12. For instance, input apparatus 24 can include any one or more of an activity monitor 32, a sonar device 36, a radar device 40, a force sensor 44, etc., without limitation, among any of a variety of other input devices such as monitors for Heart Rate (HR), Heart Rate Variability (HRV), temperature, daytime activity, and the like. The waveforms during sleep are useful for the purposes of the disclosed and claimed concept. In other words, it is not strictly necessary to calculate first sleep architecture. Output apparatus 28 includes any of a variety of output devices such as visual displays and the like, without limitation, that can visually or otherwise output instruction, the score S, the phase of the viral infection in the patient, etc.

The various input devices of input apparatus 24 provide sleep macro-architecture and micro-architecture of the patient to a number of algorithms 48 that can be considered to be among the routines 20 and which can include a machine learning algorithm. The sleep-based biometric data is processed using, for instance, a machine learning algorithm 52 or a signal processing algorithm 56, or both, to result in any one or more of the parameters set forth hereinbefore, such as TST, WASO, N3, N2, REM, and the like without limitation. These parameters can then be used as inputs to determine S in order to determine the phase of the viral infection, or the parameters, potentially also including S, can be input into the machine learning decision tree algorithm depicted generally in FIG. 4 to result in a decision as to the phase of the viral infection. It is understood that the parameters are evaluated over time and thus, over time, can include a set of baseline parameters and a set of current parameters.

Apparatus 4 displays and communicates information about inflammation levels related to the viral infection, suggests a treatments (e.g. pharmacological, anti-inflammatory) or additional tests by considering sleep changes. Further investigation involves tracking and determining a correlation between the sleep architecture and the output recovery prediction.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

What is claimed is:

1. A method of determining a phase from among a plurality of phases of a viral infection in a patient, comprising:
inputting to an algorithm a data set that comprises a set of parameters that are representative of the patient's current sleep architecture and another set of parameters that are representative of a baseline sleep architecture; and
determining the phase of the viral infection based at least in part upon the data set.

2. The method of claim 1 wherein the set of parameters that are representative of the patient's current sleep architecture comprise at least one of a current Non-Rapid Eye Movement (NREM) sleep duration, a current Rapid Eye Movement (REM) sleep duration, a current Wake After Sleep Onset (WASO) duration, and a current Total Sleep Time (TST) duration, and wherein the set of parameters that are representative of the baseline sleep architecture comprise at least one of a baseline NREM sleep duration, a baseline REM sleep duration, a baseline WASO duration, and a baseline TST duration.

3. The method of claim 2, further comprising at least one of:
receiving at least one of a current Heart Rate (HR) signal, a current Heart Rate Variability (HRV) signal, a current temperature signal, and a current electroencephalogram signal and deriving therefrom at least a portion of the set of parameters that are representative of the patient's current sleep architecture; and
receiving at least one of a baseline HR signal, a baseline HRV signal, a baseline temperature signal, and a baseline electroencephalogram signal and deriving therefrom at least a portion of the set of parameters that are representative of the baseline sleep architecture.

4. The method of claim 2, further comprising employing at least one of a ballistocardiography sensor, a Doppler radar sensor, a photoplethysmography sensor, an electroencephalogram sensor, an actigraphy sensor, and a breathing sensor to derive at least one of:
at least a portion of the set of parameters that are representative of the patient's current sleep architecture; and
at least a portion of the set of parameters that are representative of the baseline sleep architecture.

5. The method of claim 2, further comprising inputting the set of data into a machine learning device and employing the machine learning device in the determining of the phase of the viral infection.

6. The method of claim 5, further comprising employing the machine learning device to apply a set of thresholds to the set of data in the determining of the phase of the viral infection.

7. The method of claim 2, further comprising:
comparing the set of parameters that are representative of the patient's current sleep architecture with the set of parameters that are representative of the baseline sleep architecture to determine a score for the patient; and
determining the phase of the viral infection based at least in part upon the score.

8. The method of claim 7, further comprising inputting the data set and the score into a machine learning device and employing the machine learning device in the determining of the phase of the viral infection.

9. An apparatus structured to determine a phase from among a plurality of phases of a viral infection in a patient, comprising:
a processor apparatus comprising a processor and a storage;
an input apparatus structured to provide input signals to the processor apparatus;
an output apparatus structured to receive output signals from the processor apparatus;
the storage having stored therein a number of routines which, when executed on the processor, cause the apparatus to perform operations comprising:
inputting to an algorithm a data set that comprises a set of parameters that are representative of the patient's current sleep architecture and another set of parameters that are representative of a baseline sleep architecture; and
determining the phase of the viral infection based at least in part upon the data set.

10. The apparatus of claim 9 wherein the set of parameters that are representative of the patient's current sleep architecture comprise at least one of a current Non-Rapid Eye Movement (NREM) sleep duration, a current Rapid Eye Movement (REM) sleep duration, a current Wake After Sleep Onset (WASO) duration, and a current Total Sleep Time (TST) duration, and wherein the set of parameters that are representative of the baseline sleep architecture comprise at least one of a baseline NREM sleep duration, a baseline REM sleep duration, a baseline WASO duration, and a baseline TST duration.

11. The apparatus of claim 10, wherein the operations further comprise at least one of:
receiving at least one of a current Heart Rate (HR) signal, a current Heart Rate Variability (HRV) signal, a current temperature signal, and a current electroencephalogram signal and deriving therefrom at least a portion of the set of parameters that are representative of the patient's current sleep architecture; and
receiving at least one of a baseline HR signal, a baseline HRV signal, a baseline temperature signal, and a baseline electroencephalogram signal and deriving therefrom at least a portion of the set of parameters that are representative of the baseline sleep architecture.

12. The apparatus of claim 10, wherein the operations further comprise employing at least one of a ballistocardiography sensor, a Doppler radar sensor, a photoplethysmography sensor, an electroencephalogram sensor, an actigraphy sensor, and a breathing sensor to derive at least one of:
at least a portion of the set of parameters that are representative of the patient's current sleep architecture; and
at least a portion of the set of parameters that are representative of the baseline sleep architecture.

13. The apparatus of claim 10, wherein the operations further comprise inputting the set of data into a machine learning device and employing the machine learning device in the determining of the phase of the viral infection.

14. The apparatus of claim 13, wherein the operations further comprise employing the machine learning device to apply a set of thresholds to the set of data in the determining of the phase of the viral infection.

15. The apparatus of claim 10, wherein the operations further comprise:
comparing the set of parameters that are representative of the patient's current sleep architecture with the set of parameters that are representative of the baseline sleep architecture to determine a score for the patient; and
determining the phase of the viral infection based at least in part upon the score.

16. The apparatus of claim 15, wherein the operations further comprise inputting the data set and the score into a machine learning device and employing the machine learning device in the determining of the phase of the viral infection.
